(19) United States
(12) Patent Application Publication
(10) Pub. No.: US 2023/0241194 A1
(43) Pub. Date: Aug. 3, 2023

(54) PRE-ERYTHROCYTIC MALARIA VACCINES

(71) Applicants: Michael THEISEN, Copenhagen S (DK); Susheel Kumar SINGH, Copenhagen (DK); PATH, Seattle, WA (US); Sumitomo Pharma Co., Ltd., Osaka (JP)

(72) Inventors: C. Richter King, Washington, DC (US); Yimin Wu, Germantown, MD (US); Jordan Lee Plieskatt, Alexandria, VA (US); Michael Theisen, Copenhagen S (DK); Susheel Kumar Singh, Copenhagen (DK); Akihisa Fukushima, Osaka-shi, Osaka (JP)

(21) Appl. No.: 18/011,914

(22) PCT Filed: Dec. 1, 2020
(86) PCT No.: PCT/JP2020/044675

§ 371 Date: Dec. 21, 2022

Related U.S. Application Data
(60) Provisional application No. 63/045,540, filed on Jun. 29, 2020.

(51) Int. Cl. A61K 39/015; A61K 47/22; A61K 47/26; A61K 39/39; A61P 33/06

(52) U.S. Cl. CPC A61K 39/015; A61K 47/22; A61K 47/26; A61K 39/39; A61P 33/06

(57) ABSTRACT

Pre-erythrocytic malaria vaccines with good preservation stability and immunostimulatory action are provided. According the present invention, combination use of a pharmaceutical composition comprising (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide, or a pharmaceutically acceptable salt thereof, as a vaccine adjuvant with enhanced specific immune response against antigens and good preservation stability and a malaria vaccine with biological activity allow for the provision of pre-erythrocytic malaria vaccines with good preservation stability and immunostimulatory action.

### TECHNICAL FIELD

The present invention relates to combination use of a pharmaceutical composition comprising a compound, or a pharmaceutically acceptable salt thereof, useful for a vaccine adjuvant and a malaria vaccine, and a method for preventing malaria infection.

### BACKGROUND ART

Sub-unit vaccines where a part of components of a pathogen is used for an antigen can be prepared by chemical synthesis and genetic recombination, and such sub-unit vaccines are more useful than vaccines prepared from a pathogen itself in terms of safety and preparation methods of vaccines. Sub-unit vaccines, however, tend to show lower immunostimulatory action than live vaccines or inactivated vaccines prepared from a pathogen itself do. In order to enhance immunogenicity of epitopes and improve immunostimulatory action of vaccines, combination use of a vaccine antigen and an adjuvant has been studied for prevention and treatment for diseases.

Recently, (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaenamide, referred to as “Compound A” hereinafter, as shown below has been reported as an adjuvant having TLR7 agonistic activity (PTL 1).

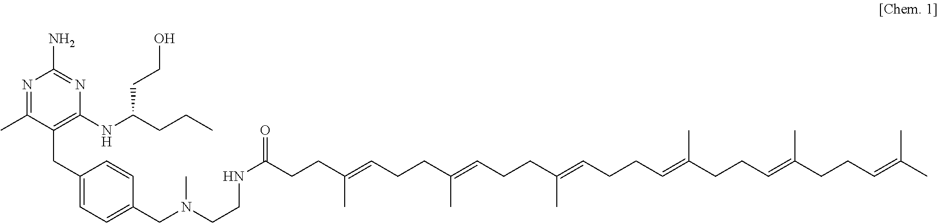 [SMILES: CCC[C@@H](CCO)Nc1nc(N)nc(C)c1Cc1ccc(CN(C)CCNC(=O)CC/C(C)=C/CC/C(C)=C/CC/C(C)=C/CC/C=C(\C)CC/C=C(\C)CCC=C(C)C)cc1]

Compound A has the good vaccine-adjuvant activity, but it is required to be formulated in a formulation such as emulsions when administered to mammals as a vaccine adjuvant. In general, it is known that emulsion formulations comprise antioxidant agents such as ascorbic acids to improve the preservation stability in formulations. It has, however, not been known that antioxidant agents such as ascorbic acids can stabilize particle-size distribution.

It has been known that a sporozoite surface protein of malaria parasites, Plasmodium falciparum, PfCSP, consisting of 397 amino acids is a target antigen for a pre-erythrocytic malaria vaccine. Due to the difficulties in the formation of correctly folded proteins, full-length PfCSP, denoted PfCSP4/38, has proven to be a difficult target for production in most heterologous expression systems (NPLs 1-6).

A known malaria vaccine acquires immunogenicity enhanced by an adjuvant, Alhydrogel® (NPL 1). Alhydrogel® is different from Compound A.

### CITATION LIST

### Patent Literature

[PTL 1] WO 2017/061532

### Non Patent Literature

[NPL 1] Gordon, D. M. et al., J Infect Dis 1995; 171, 1576-1585
[NPL 2] Young, J. F. et al., Science 1985; 228, 958-962
[NPL 3] Noe, A. R. et al., PLoS One 2014; 9, e107764
[NPL 4] Schwenk, R. et al., PLoS One 2014; 9, e111020
[NPL 5] Kedees, M. H. et al., Exp Parasitol 2002; 101, 64-68
[NPL 6] Plassmeyer, M. L. et al., J Biol Chem 2009; 284, 26951-26963
[NPL 7] Singh, S. K. et al., Microbial cell factories 2018; 17, 55
[NPL 8] Singh, S. K. et al., Microbial cell factories 2017; 16, 97
[NPL 9] Theisen, M. et al., Vaccine 2014; 32, 2623-2630

### SUMMARY OF INVENTION

### Technical Problem

The present invention provides combination use of a pharmaceutical composition comprising a compound, or a pharmaceutically acceptable salt thereof, useful for a vaccine adjuvant with good preservation stability and immunostimulatory action and a pre-erythrocytic malaria vaccine, and a method for preventing malaria infection comprising administering the pharmaceutical composition and the pre-erythrocytic malaria vaccine to mammals.

### Solution to Problem

Compound A has six unsaturated bonds derived from the intramolecular squalene-like structure. During studies of formulations of Compound A, it has been found that when Compound A is formulated into lyophilized formulations of common emulsion formulations with squalene as an oil composition, the intramolecular unsaturated bonds are oxidized, and thereby, the content of Compound A decreases. After further studies by the inventors to provide formulations of vaccine adjuvants with feasible preservation stability, improved stability against oxidation of Compound A has been achieved by using squalane as an oil composition in formulation of an emulsion composition of Compound A. In addition, formulations with good preservation stability, particularly the stability of particle-size distributions as well as the oxidative stability of Compound A itself have been achieved by addition of an antioxidant agent such as ascorbic acids.

The inventors have also found that an improved PfCSP, denoted PfCSP4/38, as a pre-erythrocytic malaria vaccine retains a conformational epitope for antibodies as confirmed by both in vivo and in vitro characterizations, and biologically active.

It is expected that combination use of a pharmaceutical composition comprising Compound A and a malaria vaccine comprising PfCSP4/38 is useful for pre-erythrocytic malaria vaccines with good preservation stability and immunostimulatory action.

Embodiments of the present invention are illustrated as follows.

Item 1. A method for preventing malaria infection, comprising administering a pharmaceutically effective amount of a combination of I) a pharmaceutical composition and II) a vaccine to a human, wherein:
I) the pharmaceutical composition comprises the following ingredients i) to vi):
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide, referred to as “Compound A” hereinafter, or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
   II) the vaccine is a malaria vaccine comprising an antigen comprising the sequence represented by SEQ ID NO: 1, SEQ ID NO: 2, SEQ ID NO: 3, or any of sequences substantially identical thereto.
   Item 2. A combination drug comprising:
I) a pharmaceutical composition comprising:
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
   II) a malaria vaccine comprising an antigen comprising the sequence represented by SEQ ID NO: 1, SEQ ID NO: 2, SEQ ID NO: 3, or any of sequences substantially identical thereto.
   Item 3. A vaccine formulation for malaria comprising:
   I) a pharmaceutical composition comprising:
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
   II) a malaria vaccine comprising an antigen comprising the sequence represented by SEQ ID NO: 1, SEQ ID NO: 2, SEQ ID NO: 3, or any of sequences substantially identical thereto.
   Item 4. A kit comprising:
   I) a pharmaceutical composition comprising:
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
   II) a malaria vaccine comprising an antigen comprising the sequence represented by SEQ ID NO: 1, SEQ ID NO: 2, SEQ ID NO: 3, or any of sequences substantially identical thereto.
   Item 5. The method, the combination drug, the vaccine formulation, or the kit according to any one of Items 1 to 4, wherein the pharmaceutical composition is an oil-in-water type emulsion formulation or a lyophilized formulation thereof.
   Item 6. The method, the combination drug, the vaccine formulation, or the kit according to any one of Items 1 to 5, wherein the hydrophilic surfactant is polyoxyethylene sorbitan fatty acid esters (e.g., polysorbate 20, polysorbate 40, polysorbate 60, polysorbate 65, and polysorbate 80); polyoxyethylene hydrogenated castor oils (e.g., polyoxyethylene hydrogenated castor oil 10, polyoxyethylene hydrogenated castor oil 20, polyoxyethylene hydrogenated castor oil 40, polyoxyethylene hydrogenated castor oil 50, and polyoxyethylene hydrogenated castor oil 60); or polyoxyethylene polyoxypropylene glycols (e.g., polyoxyethylene (42) polyoxypropylene (67) glycol, polyoxyethylene (54) polyoxypropylene (39) glycol, polyoxyethylene (105) polyoxypropylene (5) glycol, polyoxyethylene (124) polyoxypropylene (39) glycol, polyoxyethylene (160) polyoxypropylene (30) glycol, polyoxyethylene (196) polyoxypropylene (67) glycol, and polyoxyethylene (200) polyoxypropylene (70) glycol).
   Item 7. The method, the combination drug, the vaccine formulation, or the kit according to any one of Items 1 to 5, wherein the hydrophilic surfactant is polysorbate 20, polysorbate 40, polysorbate 80, polyoxyethylene hydrogenated castor oil 60, or polyoxyethylene (160) polyoxypropylene (30) glycol.
   Item 8. The method, the combination drug, the vaccine formulation, or the kit according to any one of Items 1 to 5, wherein the hydrophilic surfactant is polysorbate 20, polysorbate 40, or polysorbate 80.
   Item 9. The method, the combination drug, the vaccine formulation, or the kit according to any one of Items 1 to 8, wherein the lipophilic surfactant is sorbitan fatty acid esters (e.g., sorbitan fatty acid ester, sorbitan monolaurate, sorbitan monopalmitate, sorbitan monostearate, sorbitan tristearate, sorbitan monooleate, sorbitan sesquioleate, sorbitan trioleate, and medium-chain triglyceride); glycerin fatty acid esters (e.g., glycerin fatty acid ester, glyceryl monostearate, glyceryl monomyristate, glyceryl monooleate, and glyceryl triisooctanoate); sucrose fatty acid esters (e.g., sucrose fatty acid ester, sucrose stearate, and sucrose palmitate); or propylene glycol fatty acid esters (e.g., propylene glycol fatty acid ester and propylene glycol monostearate).
   Item 10. The method, the combination drug, the vaccine formulation, or the kit according to any one of Items 1 to 8, wherein the lipophilic surfactant is sorbitan fatty acid ester, sorbitan monooleate, sorbitan sesquioleate, or sorbitan trioleate.
   Item 11. The method, the combination drug, the vaccine formulation, or the kit according to any one of Items 1 to 8, wherein the lipophilic surfactant is sorbitan trioleate.
   Item 12. The method, the combination drug, the vaccine formulation, or the kit according to any one of Items 1 to 11, wherein the pharmaceutical composition further comprises an antioxidant agent B selected from the group consisting of tocopherols (e.g., α-tocopherol, β-tocopherol, γ-tocopherol, and δ-tocopherol); tocopherol acetate; and butylhydroxyanisole.
   Item 13. The method, the combination drug, the vaccine formulation, or the kit according to any one of Items 1 to 11, wherein the pharmaceutical composition further comprises an antioxidant agent B selected from the group consisting of α-tocopherol, β-tocopherol, γ-tocopherol, and δ-tocopherol.
   Item 14. The method, the combination drug, the vaccine formulation, or the kit according to any one of Items 1 to 11, wherein the pharmaceutical composition further comprises an antioxidant B of α-tocopherol.
   Item 15. The method, the combination drug, the vaccine formulation, or the kit according to any one of Items 1 to 14, wherein the antioxidant agent A is ascorbyl palmitate, potassium ascorbate, sodium ascorbate, or ascorbic acid.
   Item 16. The method, the combination drug, the vaccine formulation, or the kit according to any one of Items 1 to 14, wherein the antioxidant agent A is sodium ascorbate or potassium ascorbate.
   Item 17. The method, the combination drug, the vaccine formulation, or the kit according to any one of Items 1 to 16, wherein the excipient is non-reducing sugars (e.g., sucrose and trehalose) or sugar alcohols (e.g., sorbitol, erythritol, xylitol, maltitol, and lactitol).
   Item 18. The method, the combination drug, the vaccine formulation, or the kit according to any one of Items 1 to 16, wherein the excipient is sucrose, trehalose, sorbitol, or xylitol.
   Item 19. The method, the combination drug, the vaccine formulation, or the kit according to any one of Items 1 to 16, wherein the excipient is sucrose or trehalose.
   Item 20. The method, the combination drug, the vaccine formulation, or the kit according to any one of Items 1 to 19, wherein the content of squalane in the pharmaceutical composition ranges from 50- to 500-fold of the weight of Compound A.
   Item 21. The method, the combination drug, the vaccine formulation, or the kit according to any one of Items 1 to 19, wherein the content of squalane in the pharmaceutical composition ranges from 100- to 400-fold of the weight of Compound A.
   Item 22. The method, the combination drug, the vaccine formulation, or the kit according to any one of Items 1 to 19, wherein the content of squalane in the pharmaceutical composition ranges from 200- to 300-fold of the weight of Compound A.
   Item 23. The method, the combination drug, the vaccine formulation, or the kit according to any one of Items 1 to 22, wherein the content of the hydrophilic surfactant in the pharmaceutical composition ranges from 0.5- to 250-fold of the weight of Compound A.
   Item 24. The method, the combination drug, the vaccine formulation, or the kit according to any one of Items 1 to 22, wherein the content of the hydrophilic surfactant in the pharmaceutical composition ranges from 5- to 100-fold of the weight of Compound A.
   Item 25. The method, the combination drug, the vaccine formulation, or the kit according to any one of Items 1 to 22, wherein the content of the hydrophilic surfactant in the pharmaceutical composition ranges from 10- to 50-fold of the weight of Compound A.
   Item 26. The method, the combination drug, the vaccine formulation, or the kit according to any one of Items 1 to 25, wherein the content of the lipophilic surfactant in the pharmaceutical composition ranges from 0.5- to 250-fold of the weight of Compound A.
   Item 27. The method, the combination drug, the vaccine formulation, or the kit according to any one of Items 1 to 25, wherein the content of the lipophilic surfactant in the pharmaceutical composition ranges from 5- to 100-fold of the weight of Compound A.
   Item 28. The method, the combination drug, the vaccine formulation, or the kit according to any one of Items 1 to 25, wherein the content of the lipophilic surfactant in the pharmaceutical composition ranges from 10- to 50-fold of the weight of Compound A.
   Item 29. The method, the combination drug, the vaccine formulation, or the kit according to any one of Items 1 to 28, wherein the content of the antioxidant agent A in the pharmaceutical composition ranges from 0.5- to 500-fold of the weight of Compound A when calculated in terms of the weight of sodium ascorbate.
   Item 30. The method, the combination drug, the vaccine formulation, or the kit according to any one of Items 1 to 28, wherein the content of the antioxidant agent A in the pharmaceutical composition ranges from 2.5- to 250-fold of the weight of Compound A when calculated in terms of the weight of sodium ascorbate.
   Item 31. The method, the combination drug, the vaccine formulation, or the kit according to any one of Items 1 to 28, wherein the content of the antioxidant agent A in the pharmaceutical composition ranges from 5- to 100-fold of the weight of Compound A when calculated in terms of the weight of sodium ascorbate.
   Item 32. The method, the combination drug, the vaccine formulation, or the kit according to any one of Items 1 to 31, wherein the content of the excipient in the pharmaceutical composition ranges from 50- to 1000-fold of the weight of Compound A.
   Item 33. The method, the combination drug, the vaccine formulation, or the kit according to any one of Items 1 to 31, wherein the content of the excipient in the pharmaceutical composition ranges from 100- to 750-fold of the weight of Compound A.
   Item 34. The method, the combination drug, the vaccine formulation, or the kit according to any one of Items 1 to 31, wherein the content of the excipient in the pharmaceutical composition ranges from 200- to 625-fold of the weight of Compound A.
   Item 35. The method, the combination drug, the vaccine formulation, or the kit according to any one of Items 12 to 34, wherein the content of the antioxidant agent B in the pharmaceutical composition ranges from 5- to 250-fold of the weight of Compound A.
   Item 36. The method, the combination drug, the vaccine formulation, or the kit according to any one of Items 12 to 34, wherein the content of the antioxidant agent B in the pharmaceutical composition ranges from 12.5- to 125-fold of the weight of Compound A.
   Item 37. The method, the combination drug, the vaccine formulation, or the kit according to any one of Items 12 to 34, wherein the content of the antioxidant agent B in the pharmaceutical composition ranges from 25- to 50-fold of the weight of Compound A.
   Item 38. The method, the combination drug, the vaccine formulation, or the kit according to any one of Items 1 to 37, wherein the weight of Compound A ranges from 0.0001- to 0.65-fold of the weight of a lyophilized substance of the pharmaceutical composition excluding Compound A.
   Item 39. The method, the combination drug, the vaccine formulation, or the kit according to any one of Items 1 to 37, wherein the weight of Compound A ranges from 0.0002- to 0.35-fold of the weight of a lyophilized substance of the pharmaceutical composition excluding Compound A.
   Item 40. The method, the combination drug, the vaccine formulation, or the kit according to any one of Items 1 to 37, wherein the weight of Compound A ranges from 0.0005- to 0.065-fold of the weight of a lyophilized substance of the pharmaceutical composition excluding Compound A.
   Item 41. The method, the combination drug, the vaccine formulation, or the kit according to any one of Items 5 to 40, wherein the particle size D₉₀ values of an emulsion of the pharmaceutical composition right after manufacturing and an emulsion of the pharmaceutical composition reconstituted after storage for 6 months at 25° C. as a lyophilized formulation are 1000 nm or below.
   Item 42. The method, the combination drug, the vaccine formulation, or the kit according to any one of Items 5 to 41, wherein the increased amount in the area percentage value of an impurity UK-1.02 after a lyophilized formulation of the pharmaceutical composition is stored for 6 months at 5° C. is 5.0% or below.
   Item 43. The method, the combination drug, the vaccine formulation, or the kit according to any one of Items 5 to 41, wherein the increased amount in the area percentage value of an impurity UK-1.02 after a lyophilized formulation of the pharmaceutical composition is stored for 6 months at 5° C. is 1.0% or below.
   Item 44. A method for preventing malaria infection, comprising administering a pharmaceutically effective amount of I) a pharmaceutical composition to a human in combination with a pharmaceutically effective amount of II) a vaccine, wherein:
   I) the pharmaceutical composition comprises the following ingredients i) to vi):
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
   II) the vaccine is a malaria vaccine comprising an antigen comprising the sequence represented by SEQ ID NO: 1, SEQ ID NO: 2, SEQ ID NO: 3, or any of sequences substantially identical thereto.
   Item 45. A pharmaceutical composition for use in preventing malaria infection in combination with a malaria vaccine, wherein:
the pharmaceutical composition comprises the following ingredients i) to vi):
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and the malaria vaccine comprises an antigen comprising the sequence represented by SEQ ID NO: 1, SEQ ID NO: 2, SEQ ID NO: 3, or any of sequences substantially identical thereto.
   Item 46. A malaria vaccine for use in preventing malaria infection in combination with a pharmaceutical composition, wherein:
the malaria vaccine comprises an antigen comprising the sequence represented by SEQ ID NO: 1, SEQ ID NO: 2, SEQ ID NO: 3, or any of sequences substantially identical thereto; and
the pharmaceutical composition comprises the following ingredients i) to vi):
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant.

### Advantageous Effects of Invention

According to the present invention, combination use of a pharmaceutical composition as a vaccine adjuvant with enhanced specific immune response against antigens and good preservation stability and a malaria vaccine with homogeneity and biological activity allows for the provision of pre-erythrocytic malaria vaccines with good preservation stability and immunostimulatory action.

### BRIEF DESCRIPTION OF DRAWINGS

FIG. 1 shows schematic representation of PfCSP4/38 containing the full repeat region (4 NVDP/38 NANP) which is reactive to mAb 1E8. The position of the signal peptide (SP), the central repeat region containing the NVDP and NANP protein units, and glycosylphosphatidylinositol (GPI) anchor are indicated. The four C-terminal cysteines are indicated with lines and is reactive to the conformational mAb 1A6. N-terminus along with N-terminal cysteine is omitted, with sequence originating at Tyr26 and terminating prior to GPI anchor of C-terminus (Ser383). Recombinant PfCSP4/38 protein contains the vector encoded amino acid residues A-E-R-S at the N-terminal end and a short flexible linker (G-G-S) followed by a six-histidine-tag at the C-terminal end.

FIG. 2 shows electrophoresis of purified PfCSP4/38. Coomassie blue-stained 4-12.5% polyacrylamide gel with different amount (0.5, 1.0, and 1.5 μg PfCSP) in non-reduced and reduced conditions. Western blot using the same gel loading as in the upper panel with conformational (mAb 1A6) and non-conformational (mAb 1E8) antibody. The sizes (kDa) of the molecular mass markers are indicated.

FIG. 3 shows immunogenicity of PfCSP4/38. Mice (n=8) were immunized with 10 μg of purified PfCSP4/38 adjuvanted with Alhydrogel® on days 0, 21, and 42 and bleed on days 21, 42, and 56. IgG antibody titres were expressed as EC50 values. Significant differences in antibody titre between days were analyzed using Mann-Whitney Rank Sum test.

FIG. 4 shows that anti-PfCSP4/38 antibodies inhibited: (A-B) sporozoite gliding motility, (C-D) traversal and (EF) liver cell invasion. Mouse polyclonal sera containing PfCSP4/38-specific antibodies (A, C, and E) or mAb 2A10 (B, D, and F) were normalized against naïve mouse sera. The initial concentration of mouse polyclonal (PfCSP4/38) IgG in the sera was first determined by sporozoite ELISA where a standard reference curve generated with the mAb2A10 was used to convert the sera-specific optical density values to concentration values using a four-parameter curve fitting program. The assay specific IC50 concentration values were estimated using serial dilutions of the sera based on the determined initial concentration of the polyclonal PfCSP4/38 antibody present. The mAb 2A10 concentrations are from purified IgG. The IC50 was calculated by logistic regression using a four-parameter model and least square method to determine the best fit. The lowest value of each y-value was set to zero and the maximum value set to 100%. pAb: polyclonal antibody.

### DESCRIPTION OF EMBODIMENTS

### Pharmaceutical Composition

Pharmaceutical compositions herein include a lyophilized formulation of an emulsion comprising Compound A, squalane, an antioxidant agent A of ascorbic acids, and an excipient A. The emulsion formulation before lyophilization and a reconstituted emulsion formulation from the lyophilized formulation are also encompassed in the present invention.

In the pharmaceutical compositions, Compound A comprised in the active ingredient may be in the free form or any pharmaceutically acceptable acid-addition salts or base-addition salts thereof. Such acid-addition salts include, for example, acid-addition salts with inorganic or organic acids such as hydrochloric acid, hydrobromic acid, sulfuric acid, trifluoroacetic acid, citric acid, and maleic acid. Such base-addition salts include, for example, alkali metal salts such as sodium and potassium salts, alkaline-earth metal salts such as calcium salt, and ammonium salts. Compound A or a pharmaceutically acceptable salt thereof herein may also exist in the form of hydrates and solvates which are also included in Compound A or a pharmaceutically acceptable salt thereof herein. Details and preparations for them are described in PTL 1, and Compound A or a pharmaceutically acceptable salt thereof may be prepared according to, for example, the methods described in PTL 1.

The content of Compound A in the pharmaceutical composition is described as that of the free form of Compound A. When Compound A is used in its pharmaceutically acceptable salt, the content is calculated in terms of the weight of Compound A with addition of the weight of the salt.

Emulsions or emulsion formulations herein refer to oil-in-water type or water-in-oil type emulsions. Oil-in-water type emulsions are preferred. The ratio by weight of an oil composition to an aqueous solution ranges preferably from 1:99 to 15:85, more preferably from 2:98 to 10:90, furthermore preferably from 3:97 to 9:91, still furthermore preferably from 4:96 to 7:93. In an emulsion formulation herein, Compound A is dissolved to exist in the oil composition.

Lyophilized formulations herein refer to the formulation where water is removed from the emulsion formulation under lyophilization. The emulsion formulation may be reconstituted with two- to twenty-fold weight of water for injection to the weight of a lyophilized formulation.

The hydrophilic surfactant herein includes polyoxyethylene sorbitan fatty acid esters (e.g., polysorbate 20, polysorbate 40, polysorbate 60, polysorbate 65, and polysorbate 80); polyoxyethylene hydrogenated castor oils (e.g., polyoxyethylene hydrogenated castor oil 10, polyoxyethylene hydrogenated castor oil 20, polyoxyethylene hydrogenated castor oil 40, polyoxyethylene hydrogenated castor oil 50, and polyoxyethylene hydrogenated castor oil 60); and polyoxyethylene polyoxypropylene glycols (e.g., polyoxyethylene (42) polyoxypropylene (67) glycol, polyoxyethylene (54) polyoxypropylene (39) glycol, polyoxyethylene (105) polyoxypropylene (5) glycol, polyoxyethylene (124) polyoxypropylene (39) glycol, polyoxyethylene (160) polyoxypropylene (30) glycol, polyoxyethylene (196) polyoxypropylene (67) glycol, and polyoxyethylene (200) polyoxypropylene (70) glycol). Polysorbate 20, polysorbate 40, polysorbate 80, polyoxyethylene hydrogenated castor oil 60, and polyoxyethylene (160) polyoxypropylene (30) glycol are preferred; polysorbate 20, polysorbate 40, and polysorbate 80 are further preferred; and polysorbate 80 is particularly preferred.

The content of the hydrophilic surfactant in the pharmaceutical composition ranges from 0.5- to 250-fold of the weight of Compound A, preferably from 5- to 100-fold, more preferably from 10- to 50-fold.

The lipophilic surfactant herein includes sorbitan fatty acid esters (e.g., sorbitan fatty acid ester, sorbitan monolaurate, sorbitan monopalmitate, sorbitan monostearate, sorbitan tristearate, sorbitan monooleate, sorbitan sesquioleate, sorbitan trioleate, and medium-chain triglyceride); glycerin fatty acid esters (e.g., glycerin fatty acid ester, glyceryl monostearate, glyceryl monomyristate, glyceryl monooleate, and glyceryl triisooctanoate); sucrose fatty acid esters (e.g., sucrose fatty acid ester, sucrose stearate, and sucrose palmitate); and propylene glycol fatty acid esters (e.g., propylene glycol fatty acid ester and propylene glycol monostearate). Sorbitan fatty acid ester, sorbitan monooleate, sorbitan sesquioleate, and sorbitan trioleate are preferred; and sorbitan trioleate is further preferred.

The content of the lipophilic surfactant in the pharmaceutical composition ranges from 0.5- to 250-fold of the weight of Compound A, preferably from 5- to 100-fold, more preferably from 10- to 50-fold.

Oil compositions in the pharmaceutical composition herein include squalane. In formulation studies of the pharmaceutical compositions, squalane is preferably used for the oil composition in the pharmaceutical composition because the oxidative stability of Compound A is better in the use of squalane than that in the use of squalene commonly used as oil compositions for emulsion formulations. The content of squalane in the pharmaceutical composition ranges from 50- to 500-fold of the weight of Compound A, preferably from 100- to 400-fold, more preferably from 200- to 300-fold.

The antioxidant agent A herein includes ascorbic acid esters (e.g., L-ascorbyl stearate and ascorbyl palmitate); inorganic acid salts of ascorbic acid (e.g., potassium ascorbate, sodium ascorbate, and calcium ascorbate); and ascorbic acid. Ascorbyl palmitate, potassium ascorbate, sodium ascorbate, and ascorbic acid are preferred; and sodium ascorbate and potassium ascorbate are further preferred.

The content of the antioxidant agent A in the pharmaceutical composition ranges from 0.5- to 500-fold of the weight of Compound A, preferably from 2.5- to 250-fold, more preferably from 5- to 100-fold, wherein the content is calculated in terms of sodium ascorbate; i.e., the content is calculated by converting ascorbic acid of the antioxidant agent A, ascorbic acid derivatives, into sodium ascorbate by weight.

The excipient A herein includes non-reduced sugars and sugar alcohols (except for mannitol). Non-reduced sugars (e.g., sucrose, trehalose) and sugar alcohols (e.g., sorbitol, erythritol, xylitol, maltitol, and lactitol) are preferred; sucrose, trehalose, sorbitol, and xylitol are further preferred; sucrose and trehalose are furthermore preferred; and sucrose is particularly preferred.

The content of the excipient A in the pharmaceutical composition ranges from 50- to 1000-fold of the weight of Compound A, preferably from 100- to 750-fold, more preferably from 200- to 625-fold.

The antioxidant agent B herein includes tocopherols (e.g., α-tocopherol, β-tocopherol, γ-tocopherol, and δ-tocopherol); tocopherol acetate; and butylhydroxyanisole. α-tocopherol, β-tocopherol, γ-tocopherol, and δ-tocopherol are preferred; and α-tocopherol is further preferred.

The content of the antioxidant agent B in the pharmaceutical composition ranges from 5- to 250-fold of the weight of Compound A, preferably from 12.5- to 125-fold, more preferably from 20- to 50-fold, furthermore preferably from 25- to 50-fold.

Lyophilized formulations herein may be prepared by charging an emulsion into a vial and lyophilizing under commonly-used manufacturing conditions with a lyophilizer. Such manufacturing conditions are not limited, but specifically include, for example, the condition of freezing at around −40° C., followed by depressurizing in vacuo inside while increasing the temperature inside to −20° C. and drying for around 10 to 80 hours, then increasing the temperature inside to 25° C. and drying for around 10 to 30 hours.

One embodiment of the pharmaceutical compositions includes a lyophilized formulation of an emulsion, comprising:

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaenamide or a pharmaceutically acceptable salt thereof;
ii) squalane;
iii) an antioxidant agent A selected from the group consisting of ascorbic acid esters (e.g., L-ascorbyl stearate and ascorbyl palmitate), inorganic salts of ascorbic acid (e.g., potassium ascorbate, sodium ascorbate, and calcium ascorbate), and ascorbic acid;
iv) an excipient A selected from the group consisting of non-reduced sugars and sugar alcohols (except for mannitol);
v) a hydrophilic surfactant; and
vi) a lipophilic surfactant.

Another embodiment of the pharmaceutical compositions includes a lyophilized formulation of an emulsion, comprising:

i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaenamide or a pharmaceutically acceptable salt thereof;
ii) squalane;
iii) an antioxidant agent A selected from the group consisting of ascorbic acid esters (e.g., L-ascorbyl stearate and ascorbyl palmitate), inorganic salts of ascorbic acid (e.g., potassium ascorbate, sodium ascorbate, and calcium ascorbate), and ascorbic acid;
iv) an excipient A selected from the group consisting of non-reduced sugars and sugar alcohols (except for mannitol);
v) a hydrophilic surfactant such as polyoxyethylene sorbitan fatty acid esters (e.g., polysorbate 20, polysorbate 40, polysorbate 60, polysorbate 65, and polysorbate 80); polyoxyethylene hydrogenated castor oils (e.g., polyoxyethylene hydrogenated castor oil 10, polyoxyethylene hydrogenated castor oil 20, polyoxyethylene hydrogenated castor oil 40, polyoxyethylene hydrogenated castor oil 50, and polyoxyethylene hydrogenated castor oil 60); and polyoxyethylene polyoxypropylene glycols (e.g., polyoxyethylene (42) polyoxypropylene (67) glycol, polyoxyethylene (54) polyoxypropylene (39) glycol, polyoxyethylene (105) polyoxypropylene (5) glycol, polyoxyethylene (124) polyoxypropylene (39) glycol, polyoxyethylene (160) polyoxypropylene (30) glycol, polyoxyethylene (196) polyoxypropylene (67) glycol, and polyoxyethylene (200) polyoxypropylene (70) glycol);
vi) a lipophilic surfactant such as sorbitan fatty acid esters (e.g., sorbitan fatty acid ester, sorbitan monolaurate, sorbitan monopalmitate, sorbitan monostearate, sorbitan tristearate, sorbitan monooleate, sorbitan sesquioleate, sorbitan trioleate, and medium-chain triglyceride); glycerin fatty acid esters (e.g., glycerin fatty acid ester, glyceryl monostearate, glyceryl monomyristate, glyceryl monooleate, and glyceryl triisooctanoate); sucrose fatty acid esters (e.g., sucrose fatty acid ester, sucrose stearate, and sucrose palmitate); propylene glycol fatty acid esters (e.g., propylene glycol fatty acid ester and propylene glycol monostearate); and iii′) an antioxidant agent B selected from the group consisting of tocopherols (e.g., α-tocopherol, β-tocopherol, γ-tocopherol, and δ-tocopherol), tocopherol acetate, and butylhydroxyanisole.

Still another embodiment of the pharmaceutical compositions includes a lyophilized formulation comprising iii′) tocopherols (e.g., α-tocopherol, β-tocopherol, γ-tocopherol, and δ-tocopherol) without comprising the antioxidant agent A of iii) for an antioxidant agent. Such tocopherols are preferably α-tocopherol.

The content of tocopherol in the pharmaceutical composition ranges from 5- to 250-fold of the weight of Compound A, preferably from 12.5- to 125-fold, more preferably from 20- to 50-fold, furthermore preferably from 25- to 50-fold. In such a pharmaceutical composition, sodium thiosulfate or butylhydroxyanisole may also be included as an additional antioxidant agent.

The weight of Compound A in the pharmaceutical composition ranges from 0.0001- to 0.65-fold of the weight of a lyophilized substance of the pharmaceutical composition excluding Compound A, preferably from 0.0002- to 0.35-fold, more preferably from 0.0005- to 0.065-fold.

The particle size D₉₀ value of oil droplets in the pharmaceutical composition is 1000 nm or below, preferably 300 nm or below, as the particle size D₉₀ value of an emulsion during the manufacturing process or right after manufacturing. The emulsion right after manufacturing includes, for example, the emulsion within 30 seconds after manufactured. The particle size D₉₀ value of oil droplets of an emulsion of the pharmaceutical composition reconstituted after storage as a lyophilized formulation is preferably 1000 nm or below as the particle size D₉₀ value of oil droplets of an emulsion reconstituted after storage for 6 months at 5° C. or 25° C.

In the pharmaceutical compositions, the particle size D₉₀ value of oil droplets is a typical value that shows the particle-size distribution of oil droplet particles comprised in an emulsion and refers to a 90% particle size based on the scattering intensity. In general, particle size D₉₀ values are measured and calculated with a dynamic-light-scattering particle-size distribution analyzer, laser-diffraction particle-size analyzer, or image-processing particle-size distribution analyzer. The particle size D₉₀ values herein refer to those measured with a dynamic-light-scattering particle-size distribution analyzer: Zetasizer Nano ZS (Malvern Instruments).

In the pharmaceutical compositions, an impurity UK-1.02 is one of typical impurities detected in the assessment of related substances with a high-performance liquid chromatograph. In particular, it refers to the impurity detected at the 1.02-fold elution time of Compound A in the spectrographic measurement with a 220-nm wavelength by reverse-phase high-performance liquid chromatography using pure water, acetonitrile, methanol, and trifluoroacetic acid with a Phenyl-Hexyl column (Waters Xselect CSH Phenyl-Hexyl XP Column, 4.6 mm×75 mm, 2.5 μm, model number: 186006134) injecting 0.4 to 2 μg calculated as the content of Compound A. Details of the measurement conditions are as follows.

Mobile phase A: 0.1% aqueous trifluoroacetic acid solution

Mobile phase B: acetonitrile/methanol mixed solution (8:2) containing 0.06% trifluoroacetic acid

Gradient conditions:

TABLE 1

| | Mobile phase A: |
|---|---|
| Time (min.) | Mobile phase B |
| 0.0 to 0.5 | 6:4 |
| 0.5 to 50.5 | 6:4 -> 1:9 |
| 50.5 to 65.0 | 1:9 |
| 65.0 to 65.1 | 1:9 -> 6:4 |
| 75.0 | 6:4 |

Flow rate: 0.5 mL/min.

Column temperature: Constant temperature at around 40° C.

The preservation stability of the pharmaceutical composition means that the increased amount in the area percentage value of an impurity UK-1.02 after a lyophilized formulation of the pharmaceutical composition is stored for 6 months at 5° C. is 5.0% or below, preferably 1.0% or below, of the value at the start of storage. The area percentage values are compared in the actual measured values.

The pharmaceutical composition is stored in the lyophilized condition where the oil-in-water emulsion prepared is emulsified, followed by aseptic filtration with an aseptic filtration filter. In the aseptic filtration, the particle size D₉₀ value is preferably 1000 nm or below so as to avoid clogging and allow for efficient filtration.

The pharmaceutical composition may further comprise additional additives as long as the particle size of the emulsion after reconstitution is unchanged. When administered, the pharmaceutical composition may be administered in combination with a formulation comprising a vaccine antigen, also referred to as a “vaccine” herein, as long as the particle size of the emulsion after reconstitution is unchanged. Mixing methods and ratios of the pharmaceutical composition and such a vaccine antigen are not limited, but for example, a formulation comprising a vaccine antigen may be combined by inversion mixing in a vial in the same volume as that of the reconstituted emulsion formulation.

The pharmaceutical composition may be provided as a kit comprising a lyophilized formulation comprising Compound A and a vaccine antigen.

The pharmaceutical composition may be administered by reconstitution with 2- to 20-fold of water for injection by weight of a lyophilized formulation when administered, followed by mixing with a formulation comprising a vaccine antigen. A dosage amount of the pharmaceutical composition is 1 ng to 250 mg, preferably 1 ng to 50 mg, of the weight of Compound A per dose. The administration may be in a single dose or with one or more additional doses depending on the kind of the vaccine antigen simultaneously administered or the age of the subject to be administered.

The preservation stability of pharmaceutical compositions was assessed according to the test examples below.

### Vaccine Antigen

A vaccine antigen herein may be an antigen comprising the sequence represented by SEQ ID NO: 1, SEQ ID NO: 2, SEQ ID NO: 3, or any of sequences substantially identical thereto. The term “substantially identical” sequence herein means any sequences sharing at least 70%, 75%, 80%, 85%, 90%, 95%, 96%, 97%, 98%, or 99% homology with the sequence represented by SEQ ID NO: 1, SEQ ID NO: 2, or SEQ ID NO: 3. One embodiment of the vaccine antigen in the present invention includes a malaria vaccine comprising the full-length PfCSP antigen. In another embodiment, the vaccine antigen is a vaccine comprising an antigen of histidine-tagged PfCSP4/38 having the sequence represented by SEQ ID NO: 1 wherein any of the four amino acid residues, A-E-R-S, at the N-terminal end and/or any of the nine amino acid residues, G-G-S-H-H-H-H-H-H, at the C-terminal end may be optionally modified, deleted, or replaced, or at least one amino acid may be optionally inserted into these residues. In still another embodiment, the vaccine antigen is a vaccine comprising an antigen of PfCSP4/38 having the sequence represented by SEQ ID NO: 2 wherein any of the four amino acid residues, A-E-R-S, at the N-terminal end and/or any of the three amino acid residues, G-G-S, at the C-terminal end may be optionally modified, deleted, or replaced, or at least one amino acid may be optionally inserted into these residues. In still another embodiment, the vaccine antigen is a vaccine comprising an antigen of PfCSP4/38 comprising the sequence represented by SEQ ID NO: 3, corresponding to PfCSP₂₆₋₃₈₃.

A scalable Lactococcus lactis expression system may be used to express the PfCSP4/38 construct, which is subsequently purified and analysed. PfCSP4/38 may retain a conformational epitope for antibodies as confirmed by both in vivo and in vitro characterizations, resulting in functional immunogenicity similar to a native molecule (Test examples 4-7).

### Combination Drug/Combination Therapy

In pre-erythrocytic malaria vaccines in the present invention, combination use of a vaccine antigen and a pharmaceutical composition comprising an adjuvant, Compound A, having a TLR7 agonist activity may enhance the inducing property of IgG2 (Th1) antibody to show an improved vaccine activity. Such combination use may include administering the vaccine antigen and the pharmaceutical composition simultaneously or separately with a prescribed time interval. In one embodiment, such simultaneous combination use includes a combination drug comprising the vaccine antigen and the pharmaceutical composition. Such a combination drug may also be referred to as an “adjuvant formulation” or a “vaccine formulation” herein. In another embodiment, the combination use includes a kit comprising the vaccine antigen and the pharmaceutical composition.

Administration routes of a pharmaceutical composition, a vaccine antigen, a combination drug, and a kit herein may be selected depending on conditions such as diseases, conditions of subjects, and target sites. Such administration routes include, for example, parenteral administration, specifically, intravascular such as intravenous, subcutaneous, intracutaneous, intramuscular, transnasal, and transdermal administration. Dosage forms of a pharmaceutical composition, a vaccine antigen, and a combination drug herein include, for example, injections such as prefilled syringes.

Doses, dosage regimens, and time required for each administration of adjuvant formulations herein may be selected depending on conditions such as ages of subjects and target sites. Such adjuvant formulations may be administered or innoculated once, or may be further administered in a prescribed time period after first administration. The time period from the first administration to additional administration may, for example, be any period from 20 days to 3 years, preferably from 3 months to 2 years, more preferably from 6 months to 1 year, but is not limited thereto.

A dosage amount of a pre-erythrocytic malaria vaccine antigen per each dose in combination use herein may range from 1 μg to 200 μg, preferably from 10 μg to 30 μg, more preferably 15 μg, but is not limited thereto. One dose of an adjuvant formulation includes, for example, 0.5 mL.

A dosage amount of a pharmaceutical composition comprising Compound A per each dose in combination use herein may range from 1 ng to 250 mg, preferably from 1 ng to 50 mg, of the weight of Compound A, but is not limited thereto.

### EXAMPLES

Hereinafter, the present invention is illustrated with Examples, Reference examples, Comparative examples, and Test examples, but is not intended to be limited thereto.

Herein, “squalane (Wako pure chemical)”, “squalane (Kishimoto Special Liver Oil Co., Ltd.)”, or “squalane (Maruha Nichiro)” was used for squalane; “squalene (Wako pure chemical)”, “squalene (Kishimoto Special Liver Oil Co., Ltd.)”, or “squalene (Maruha Nichiro)” was used for squalene; “sodium ascorbate (Wako pure chemical)” or “sodium L-ascorbate (Kyowa Pharma Chemical Co., Ltd.)” was used for sodium ascorbate; “α-tocopherol (Mitsubiti-Chemical Foods Corporation)” or “all-rac-α-Tocopherol EMPROVE (registered trade mark) ESSENTIAL Ph Eur,BP,IUSP,E 307 (Merck)” was used for α-tocopherol; “Span85 (Sigma-Aldrich)”, “Rheodol SP-O30V (Kao Chemicals)”, or “Span85 (CRODA)” was used for sorbitan trioleate; “PS80 (GS) (NOF Corporation)”, “Polysorbate 80 (HX2) (NOF Corporation)”, “Tween 80 (Merck)”, or “Tween 80 HP-LQ-(HM) (CRODA)” was used for polysorbate 80; “sucrose (Nacalai Tesque)” or “sucrose low in endotoxins suitable for use as excipient EMPROVE (registered trade mark) exp Ph Eur,BP,JP,NF (Merck)” was used for sucrose; “OTSUKA distilled water for injection (Otsuka Pharmaceutical Factory)” was used for water for injection; ascorbyl palmitate, butylhydroxyanisole, and sodium thiosulfate were prepared from Wako pure chemical for use.

Abbreviations

BCA: bicinchoninic acid

CV: column volume

Da: Dalton

DNA: deoxyribonucleic acid

ELISA: enzyme-linked immunosorbent assay

EPA: Pseudomonas aeruginosa exoprotein A

HPLC: high performance liquid chromatography

kDa: kilodalton

LDS: Lithium dodecyl sulfate

MES: 2-(N-morpholino) ethanesulfonic acid

MOI: multiplicity of infection

MS: mass spectrometry

Ni-NTA: Nickel-nitrilotriacetic acid

SDS: sodium dodecyl sulfate

SDS-PAGE: sodium dodecyl sulfate polyacrylamide gel

electrophoresis

SE: size exclusion

SEC: size exclusion chromatography

SMFA: standard membrane feeding assay

### Preparation of Lyophilized Compositions

Examples 1 to 16, Comparative examples 1 to 3 Compound A was dissolved in oil-based components so as to be prepared in the compositions of Tables 1 to 4. The oil-based components are as follows: squalane, sorbitan trioleate, and α-tocopherol (Examples 1 to 9 and 15); squalane and sorbitan trioleate (Examples 10 to 13); squalane, sorbitan trioleate, α-tocopherol, and ascorbyl palmitate (Example 14); squalane, sorbitan trioleate, α-tocopherol, and butylhydroxyanisole (Example 16); squalene, sorbitan trioleate, and α-tocopherol (Comparative examples 1 to 3). Aqueous components (sucrose and polysorbate 80 in Examples 1 to 3, 14, and 16; sucrose, polysorbate 80, and sodium ascorbate in Examples 4 to 13; sucrose, polysorbate 80, and sodium thiosulfate in Example 15) were dissolved in water for injection so as to be prepared in the compositions of Tables 1 to 4, and then thereto added the above-mentioned oil-based composition. The mixture was mixed preliminarily, and emulsified to be dispersed with a ultrahigh-pressure homogenizer. The resultant was filtered through a 0.2-μm sterilizing filter, and then charged into a glass vial per 1 mL for lyophilization. Each vial was purged with nitrogen gas at ordinary pressure, and then sealed with a rubber plug to give each lyophilized composition, Examples 1 to 16 and Comparative examples 1 to 3.

TABLE 1
Table 2

| Component | Ex. 1 | Ex. 2 | Ex. 3 | Ex. 4 | Ex. 5 | Ex. 6 | Ex. 7 |
|---|---|---|---|---|---|---|---|
| Compound A | 1 | 1 | 1 | 1 | 1 | 1 | 1 |
| squalane | 225 | 200 | 237.5 | 225 | 225 | 225 | 225 |
| α-tocopherol | 25 | 50 | 12.5 | 25 | 25 | 25 | 25 |
| sodium | 0 | 0 | 0 | 2.5 | 5 | 10 | 20 |
| ascorbate | | | | | | | |
| ascorbyl | 0 | 0 | 0 | 0 | 0 | 0 | 0 |
| palmitate | | | | | | | |
| sorbitan | 25 | 25 | 25 | 25 | 25 | 25 | 25 |
| trioleate | | | | | | | |
| polysorbate 80 | 25 | 25 | 25 | 25 | 25 | 25 | 25 |
| sucrose | 500 | 500 | 500 | 500 | 500 | 500 | 500 |

Note:
Each value means the weight ratio to 1 part by weight of Compound A.

TABLE 2
Table 3

| | | | Ex. | Ex. | Ex. | Ex. | Ex. |
|---|---|---|---|---|---|---|---|
| Component | Ex. 8 | Ex. 9 | 10 | 11 | 12 | 13 | 14 |
| Compound A | 1 | 1 | 1 | 1 | 1 | 1 | 1 |
| squalane | 225 | 225 | 250 | 250 | 250 | 250 | 225 |
| α-tocopherol | 25 | 25 | 0 | 0 | 0 | 0 | 25 |
| sodium ascorbate | 40 | 60 | 10 | 20 | 40 | 60 | 0 |
| ascorbyl | 0 | 0 | 0 | 0 | 0 | 0 | 0.5 |
| palmitate | | | | | | | |
| sorbitan trioleate | 25 | 25 | 25 | 25 | 25 | 25 | 25 |
| polysorbate 80 | 25 | 25 | 25 | 25 | 25 | 25 | 25 |
| sucrose | 500 | 500 | 500 | 500 | 500 | 500 | 500 |

Note:
Each value means the weight ratio to 1 part by weight of Compound A.

TABLE 4
Table 3:

| Component | Ex. 15 | Ex. 16 |
|---|---|---|
| Compound A | 1 | 1 |
| squalane | 225 | 225 |
| α-tocopherol | 25 | 25 |
| sodium ascorbate | 0 | 0 |
| sodium thiosulfate | 5 | 0 |
| butylhydroxyanisole | 0 | 0.25 |
| sorbitan trioleate | 25 | 25 |
| polysorbate 80 | 25 | 25 |
| sucrose | 500 | 500 |

Note:
Each value means the weight ratio to 1 part by weight of Compound A.

TABLE 5
Table 4:

| | Comparative | Comparative | Comparative |
|---|---|---|---|
| Component | example 1 | example 2 | example 3 |
| Compound A | 1 | 1 | 1 |
| squalene | 237.5 | 225 | 200 |
| squalane | 0 | 0 | 0 |
| α-tocopherol | 12.5 | 25 | 50 |
| sodium ascorbate | 0 | 0 | 0 |
| sorbitan trioleate | 25 | 25 | 25 |
| polysorbate 80 | 25 | 25 | 25 |
| sucrose | 500 | 500 | 500 |

Note:
Each value means the weight ratio to 1 part by weight of Compound A.

### [Test Example 1] Assessment for Particle Sizes in the Manufacturing Process

In the manufacturing process of a lyophilized composition, the particle-size distribution of the oil droplet particles comprised in the emulsion after emulsification and before lyophilization was measured according to the following method. Emulsions were diluted with water for injection to 10-fold, and the 90% particle size (D₉₀) on the basis of the scattering intensity was measured with a dynamic-light-scattering particle-size distribution analyzer (Zetasizer Nano ZS). D₉₀ (nm) for Examples 1 to 16 and Comparative examples 1 to 3 are shown in Table 5.

TABLE 5
Table 6

| | Ex. 1 | Ex. 2 | Ex. 3 | Ex. 4 |
|---|---|---|---|---|
| D₉₀ (nm) | 294 | 276 | 274 | 273 |
| | Ex. 5 | Ex. 6 | Ex. 7 | Ex. 8 |
| D₉₀ (nm) | 287 | 293 | 270 | 250 |
| | Ex. 9 | Ex. 10 | Ex. 11 | Ex. 12 |
| D₉₀ (nm) | 259 | 280 | 288 | 279 |
| | Ex. 13 | Ex. 14 | Ex. 15 | Ex. 16 |
| D₉₀ (nm) | 266 | 289 | 290 | 295 |

| | Comparative | Comparative | Comparative |
|---|---|---|---|
| | example 1 | example 2 | example 3 |
| D₉₀ (nm) | 280 | 309 | 273 |

### [Test Example 2] Assessment for Stability (1)

The particle-size distributions for the lyophilized compositions prepared were measured at the start of storage and after 6-month storage in a constant-temperature room at 5° C. and 25° C. according to the following method. 1 mL of water for injection was added to each vial of the lyophilized compositions prepared in Examples 1 to 16 and Comparative examples 1 to 3 for reconstitution. Then, 100 μL of the reconstituted solution was taken by micropipette and mixed with 900 μL of water for injection. Then, the 90% particle size (D₉₀) on the basis of the scattering intensity was measured with a dynamic-light-scattering particle-size distribution analyzer (Zetasizer Nano ZS). D₉₀ (nm) for Examples 1 to 16 and Comparative examples 1 to 3 before and after storage are shown in Table 6.

TABLE 6
Table 7

| | Ex. 1 | Ex. 2 | Ex. 3 | Ex. 4 |
|---|---|---|---|---|
| At the start | 655 | 915 | 738 | 594 |
| 5° C. 6 M | 598 | 618 | 553 | 575 |
| 25° C. 6 M | 3300 | 1810 | 4650 | 772 |
| | Ex. 5 | Ex. 6 | Ex. 7 | Ex. 8 |
| At the start | 586 | 624 | 594 | 565 |
| 5° C. 6 M | 590 | 792 | 558 | 529 |
| 25° C. 6 M | 599 | 946 | 606 | 502 |
| | Ex. 9 | Ex. 10 | Ex. 11 | Ex. 12 |
| At the start | 564 | 662 | 621 | 562 |
| 5° C. 6 M | 600 | 610 | 601 | 508 |
| 25° C. 6 M | 937 | 594 | 551 | 488 |
| | Ex. 13 | Ex. 14 | Ex. 15 | Ex. 16 |
| At the start | 554 | 665 | 698 | 694 |
| 5° C. 6 M | 544 | 508 | 585 | 576 |
| 25° C. 6 M | 477 | 505 | 837 | 1150 |

| | Comparative | Comparative | Comparative |
|---|---|---|---|
| | example 1 | example 2 | example 3 |
| At the start | 675 | 552 | 1020 |
| 5° C. 6 M | 579 | 631 | 625 |
| 25° C. 6 M | 5880 | 969 | 1040 |

In the test results, Examples 4 to 14 formulations containing ascorbic acids as an antioxidant agent showed higher stability of the particle-size distribution after the 6-month storage in a constant temperature room at 5° C. and 25° C. with few changes from the values at the start of storage.

### [Test Example 3] Assessment for Stability (2)

The impurities amounts (the area percentage value of Uk-1.02) were measured at the start of storage and after 6-month storage in a constant temperature room at 5° C. according to the following method. The amounts were detected by spectrographic measurement with a 220-nm wavelength by reverse-phase high-performance liquid chromatography using pure water, acetonitrile, methanol, and trifluoroacetic acid with a Phenyl-Hexyl column (Waters Xselect CSH Phenyl-Hexyl XP Column, 4.6 mm×75 mm, 2.5 μm, model number: 186006134) injecting 0.4 to 2 μg as the content of Compound A. The details of the measurement conditions are as follows.

Mobile phase A: 0.1% aqueous trifluoroacetic acid solution

Mobile phase B: acetonitrile/methanol mixed solution (8:2) containing 0.06% trifluoroacetic acid

Gradient conditions:

TABLE 8

| | Mobile phase A: |
|---|---|
| Time (min.) | Mobile phase B |
| 0.0 to 0.5 | 6:4 |
| 0.5 to 50.5 | 6:4 -> 1:9 |
| 50.5 to 65.0 | 1:9 |
| 65.0 to 65.1 | 1:9 -> 6:4 |
| 75.0 | 6:4 |

Flow rate: 0.5 mL/min.

Column temperature: Constant temperature at around 40° C.

The area percentage values of the impurities peak (Uk-1.02) detected at the 1.02-fold elution time of Compound A were calculated by the following equation with the peak areas and elution times measured in this method.

Area percentage value of Uk−1.02(%)=Peak area of Uk−1.02/Total peak area of related substances and Compound A×100

The area percentage values (%) of the impurities peak (Uk-1.02) in Examples 1 to 16 and Comparative examples 1 to 3 before and after storage are shown in Table 7.

TABLE 7
Table 9

| | Ex. 1 | Ex. 2 | Ex. 3 | Ex. 4 |
|---|---|---|---|---|
| At the start | 1.3 | 0.9 | <0.1 | 0.9 |
| 5° C. 6 M | 2.4 | 2.4 | 4.6 | 1.0 |
| | Ex. 5 | Ex. 6 | Ex. 7 | Ex. 8 |
| At the start | 0.8 | 0.9 | 0.8 | <0.1 |
| 5° C. 6 M | 0.9 | 0.9 | 0.8 | 0.6 |
| | Ex. 9 | Ex. 10 | Ex. 11 | Ex. 12 |
| At the start | 0.6 | <0.1 | <0.1 | <0.1 |
| 5° C. 6 M | 0.5 | 0.3 | 0.3 | 0.4 |
| | Ex. 13 | Ex. 14 | Ex. 15 | Ex. 16 |
| At the start | <0.1 | 0.7 | 1.7 | 1.4 |
| 5° C. 6 M | 0.4 | 0.8 | 3.7 | 2.8 |

| | Comparative | Comparative | Comparative |
|---|---|---|---|
| | example 1 | example 2 | example 3 |
| At the start | 1.0 | 0.8 | 1.1 |
| 5° C. 6 M | 27.1 | 9.4 | 7.7 |

In the test results, Examples 4 to 14 formulations containing ascorbic acids as the antioxidant agent showed higher preservation stability with lower values of impurities (the area percentage value of Uk-1.02) after the 6-month storage in a constant-temperature room at 5° C. In comparison between Examples 1 to 3 formulations containing squalane as the oil-based component and Comparative examples 1 to 3 formulations containing squalene as the oil-based component, the formulations comprising squalane as the oil-based component had lower impurities values (the area percentage value of Uk-1.02), which shows that squalane contributes to the antioxidant stability of Compound A and the formulation comprising squalane may show high reservation stability.

### Preparation of Vaccine Antigens

### [Example 17] Preparation and Purification of PfCSP4/38 Lactococcus lactis Expression Construct (PfCSP4/38)

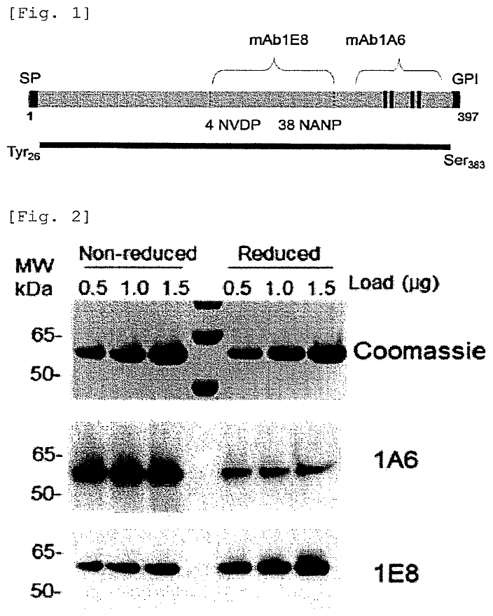

A gram-positive Lactococcus lactis, a well-established host for heterologous expression of disulfide-bonded proteins (NPLs 7-9), was used for the production of a recombinant PfCSP4/38 containing four cysteines and the full 38 NANP and 4 NVDP repeats. Codon optimized PfCSP26-383 containing 4 NDVP and 38 NANP repeats (NCBI Reference Sequence: XM_001351086.1) 3D7 synthesized by (GeneArt® Life Technologies, Germany) and inserted into pSS1 plasmid vector for protein expression in L. lactis. The final construct, containing a six-histidine-tag separated by three amino acids (GGS) at its C-terminus and denoted PfCSP4/38, was verified by sequencing and transformed into L. lactis MG1363 by electroporation (FIG. 1, SEQ ID NO: 1).

Expression and purification of PfCSP4/38

Screening for expression of PfCSP4/38 protein in L. lactis MG1363 was done. Briefly, L. lactis MG1363/PfCSP4/38 construct was grown overnight at 30° C. in 5 mL LAB medium supplemented with 4% glycerol-phosphate, 5% glucose and 1 μg/mL erythromycin. Culture supernatants were clarified by centrifugation at 9,000 g for 30 min at 4° C. and analyzed by Coomassie stained SDS-PAGE gel and western blotting. Fermentation of L. lactis MG1363/PfCSP4/38 was performed in a 1 L lab scale bioreactor (BIOFLO 310, New Brunswick Scientific) at 30° C. with gentle stirring (150 rpm) overnight with pH maintained at 6.5±0.2. Cell-free culture-filtrates were concentrated ten-fold and buffer exchanged into HEPES buffer pH 7.0 (20 mM HEPES, 50 mM NaCl, 10 mM Imidazole) using a QuixStand Benchtop system (Hollow fiber cartridge with cutoff at 30,000 Da, surface area 650 cm², GE Healthcare, Sweden). Buffer exchanged material was applied to a 5 mL Ni+2− NTA Column (HisTrap HP, GE Healthcare, Sweden). Bound protein was eluted via step gradient with 700 mM imidazole in HEPES buffer pH 7.0 (20 mM HEPES, 50 mM NaCl) at a flow rate of 4 mL/min. Eluent fractions were analyzed for purity by SDS-PAGE, pooled and further applied to a 5 mL cation exchange column (HiTrap SP HP column, GE Healthcare, Sweden) for polishing and removing host cell protein. Bound protein from IEC column was eluted through step gradient elution in HEPES buffer pH 7.0 (20 mM HEPES, 1 M NaCl and 1 mM EDTA) and fractions containing pure PfCSP4/38 were concentrated by a VIVA spin column 10 kDa cutoff (Sartorius, UK), in 20 mM HEPES, 200 mM NaCl and 1 mM EDTA, pH 7.0 and frozen at −80° C. Fractions were pooled based on SDSPAGE and immune blotting analysis with 1A6 and 1E8.

Protein Concentration Determination

Protein concentration was measured by the BCA protein assay (Thermo Fisher Scientific, USA) and endotoxin content was quantified by Pierce LAL Chromogenic Endotoxin Quantitation Kit (Thermo Fisher Scientific, USA).

### [Test Example 4] SDS-PAGE and Immune Blotting

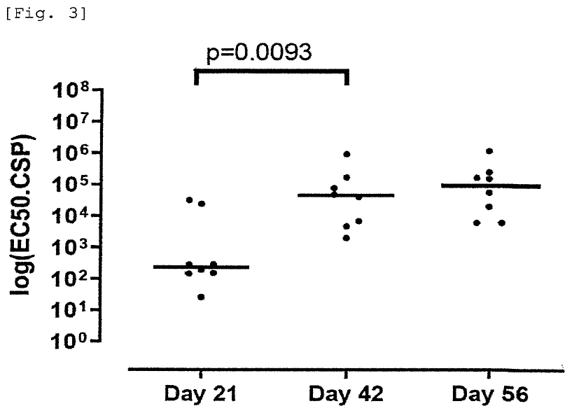

Samples were diluted with 6×SDS (Sodium dodecyl sulfate, Sigma-Aldrich) sample buffer, heated for 8 min at 98° C. and loaded in a final volume of 20 μL/well on SDS-PAGE gels (4-12% NuPAGE Bis-Tris, Invitrogen). Gels were run at 150-200 V for 50 min in 1×MOPS SDS running buffer and stained with either Coomassie or InstantBlue protein stain (Expedeon, UK). Following SDS-PAGE, proteins were transferred onto a nitrocellulose membrane for Western blot with Anti-His-HRP antibody (Miltenyi Biotech, Germany), or monoclonal antibodies 1A6 and 1E8. The conformational (1A6) and non-conformational (1E8) monoclonal antibodies were raised from immunization with a full-length PfCSP manufactured by Gennova Biopharmaceuticals (Pune, India) and kindly provided for this study by PATH, USA. Membranes were blocked in 1% skim milk in Tris buffered saline containing 0.05% Tween-20 (TBST) at room temperature for one hour. Primary antibody at a 1 μg/ml of mAb 1A6 or 1E8 (2.0 mg/ml) in TBST was added and incubated for 1 h at room temperature. Membranes were washed with TBST (3× for 5 min) and secondary antibody, 1:4,000 dilution of goat anti-mouse IgG-HRP conjugated (DAKO, Denmark) in TBST was incubated at room temperature for 1 h. Membranes were again washed with TBST (3× for 5 min), developed using HRP kit (SERA CARE, USA) (FIG. 2).

### [Test Example 5] Animals and Immunogenicity Studies and Antibody Measurements

Two in vivo animal studies were conducted in mice. For the first study, Female 6-8 week CD-1 mouse (Taconic, Denmark) kept in the Laboratory Animal Facility Center at Panum, University of Copenhagen, Denmark for, seven days before the first immunization. All procedures regarding animal immunizations complied with European and National regulations. Groups of eight mice were immunized by the s.c. route three times at three-week intervals with 10 μg of PfCSP4/38 absorbed to Alhydrogel®. Vaccine formulations were made immediately prior to use. Responses were measured using sera taken two weeks after the third immunization (Day 56).

Levels of plasma antibodies to PfCSP4/38 were measured by enzyme-linked immunosorbent assay (ELISA). Briefly, microtiter plates were coated with 0.33 μg/mL of recombinant protein and incubated with diluted samples (1:200). Bound antibody was detected with HRP-conjugated goat-anti mice IgG-HRP (DAKO, Denmark) (FIG. 3).

Immunogenicity may also be measured in a similar manner using 8110 μg of the composition of Example 6 as an adjuvant (which corresponds to 10 μg of Compound A), instead of using Alhydrogel®.

### [Test Example 6] In Vitro Sporozoite Gliding Motility Assay

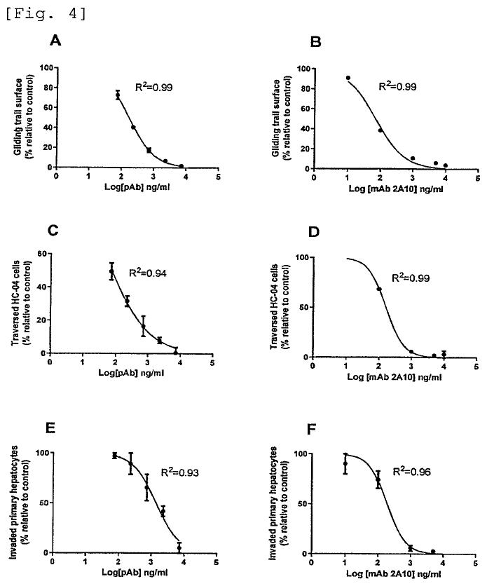

Flat-bottom optical-bottom 96-well plates with cover glass base were incubated overnight at 4° C. with an anti-PfCSP monoclonal antibody (3SP2, obtained from Radboudumc, Nijmegen). During blocking, five dilutions of 2A10 or polyclonal mouse sera (containing PfCSP4/38 antibodies) were pre-incubated with sporozoites and thereafter transferred in duplicate onto the plate. Following a 10 min spin at 3000 rpm, the sporozoites were incubated for 90 min at 37° C./5% CO₂ on the plate, and thereafter gliding motility was measured. Results were plotted in GraphPad Prism version 5.03. The number of pixels present on a stitched image made from 25 individual pictures taken per well is a measure of the amount of shed PfCSP4/38 in that particular well and therefore, differences in the number of pixels can be interpreted as differences in sporozoite gliding trail surface (FIG. 4A-4B).

### [Test Example 7] In Vitro Sporozoite Traversal and Infectivity Assay of a Human Hepatoma Cell Line

The HC-04 human hepatoma cell line was acquired through MR4 as part of the Biodefense and Emerging Infections Research Resources Repository (BEI Resources) and cultured. Traversal was conducted using freshly dissected P. falciparum NF54 sporozoites. Briefly, sporozoites were pre-incubated for 30 minutes with the monoclonal 2A10 or mouse polyclonal sera containing PfCSP4/38 antibodies. Sporozoite/antibody samples were added in duplicate to HC-04 cells seeded on 384-well plates, along with tetramethylrhodamine (Rh) labelled dextran (10,000 sporozoites and 12,500 HC-04 cells per well). Sporozoites were allowed to traverse HC-04 cells for 2 h at 37° C. in 5% CO₂ and were then washed in PBS. The level of fluorescence was measured in a Biotek Synergy 2. Data analysis was performed in GraphPad version 5.03. Traversal inhibition was normalized against the assay controls and the IC50 was calculated by logistic regression using a four parameter model and least square method to determine the best fit (FIG. 4C-4D). In vitro primary human hepatocyte invasion and maturation was performed, with a few adaptations. Cryopreserved primary human hepatocytes were obtained from Tebu-bio (donor HC10-10) and they were thawed and seeded at a density of 50,000 cells per well in a collagen coated 96-well clear bottom black plate for 2 days. Per well, 50,000 freshly dissected PfNF54 sporozoites were mixed with monoclonal 2A10 or mouse polyclonal sera containing PfCSP4/38 antibodies for 30 min, after which the mixtures were transferred onto the hepatocytes. After a quick spin (10 min at 1900 g) the plate was transferred to 37° C. in 5% CO₂. Three hours post-invasion, the medium was refreshed with normal hepatocyte culture medium. Samples were tested in duplicate with daily hepatocyte culture medium refreshments. Four days after sporozoite invasion, hepatocytes were fixed with 4% (v/v) paraformaldehyde. Wells were stained with rabbit anti-PfHSP70, followed by AlexaFluor594-labeled goat anti-rabbit IgG and 4′,6-diamidino-2-phenylindole (DAPI). By using automated high content imaging on a Cytation (BioTek) and FIJI imaging software, both the total number of hepatocytes and the number of positively-stained (infected) hepatocytes were determined. Data analysis was performed in GraphPad version 5.03. Invasion inhibition was normalized against the assay controls and the IC50 was calculated by logistic regression using a four parameter model and least square method to determine the best fit (FIG. 4E-4F).

### INDUSTRIAL APPLICABILITY

The pharmaceutical composition comprising Compound A, or a pharmaceutically acceptable salt thereof, can show high preservation stability and immunostimulatory action as a vaccine adjuvant, and combination use of the composition with a malaria vaccine may be useful for preventing malaria infection.

[Sequence Listing Free Text]
SEQ ID NO: 1
aersygsss ntrvlnelny dnagtnlyne lemnyygkqe
nwysikknsr slgenddgnn edneklrkpk hkklkqpadg
npdpnanpnv dpnanpnvdp nanpnvdpna npnanpnanp
nanpnanpna npnanpnanp nanpnanpna npnanpnanp
nanpnanpna npnanpnvdp nanpnanpna npnanpnanp
nanpnanpna npnanpnanp nanpnanpna npnanpnanp
nanpnanpna npnknnqgng qghnmpndpn rnvdenanan
savknnnnee psdkhikeyl nkiqnslste wspcsvtcgn
giqvrikpgs ankpkdeldy andiekkick mekcssvfnv
vnsggshhhhhh
SEQ ID NO: 2
aersygsss ntrvlnelny dnagtnlyne lemnyygkqe
nwysikknsr slgenddgnn edneklrkpk hkklkqpadg
npdpnanpnv dpnanpnvdp nanpnvdpna npnanpnanp
nanpnanpna npnanpnanp nanpnanpna npnanpnanp
nanpnanpna npnanpnvdp nanpnanpna npnanpnanp
nanpnanpna npnanpnanp nanpnanpna npnanpnanp
nanpnanpna npnknnqgng qghnmpndpn rnvdenanan
savknnnnee psdkhikeyl nkiqnslste wspcsvtcgn
giqvrikpgs ankpkdeldy andiekkick mekcssvfnv
vnsggs
SEQ ID NO: 3
ygsss ntrvlnelny dnagtnlyne lemnyygkqe
nwysikknsr slgenddgnn edneklrkpk hkklkqpadg
npdpnanpnv dpnanpnvdp nanpnvdpna npnanpnnan
pnanpnaanp npnanpnanp nanpnanpna npnanpnanp
nanpnanpna npnanpnvdp nanpnanpna npnanpnanp
nanpnanpna npnanpnanp nanpnanpna npnanpnanp
nanpnanpna npnknnqgng qghnmpndpn rnvdenanan
savknnnnee psdkhikeyl nkiqnslste wspcsctcgn
giqvrikpgs ankpkdeldy andiekkick mekcssvfnv
vns
[Sequence Listing]
675935 SEQ.txt

1. A method for preventing malaria infection, comprising administering a pharmaceutically effective amount of a combination of I) a pharmaceutical composition and II) a vaccine to a human, wherein:
I) the pharmaceutical composition comprises the following ingredients i) to vi):
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
II) the vaccine is a malaria vaccine comprising an antigen comprising the sequence represented by SEQ ID NO: 1, SEQ ID NO: 2, SEQ ID NO: 3, or any of sequences substantially identical thereto.

2. A combination drug comprising:
I) a pharmaceutical composition comprising:
i) (4E,8E,12E,16E,20E)-N-{2-[{4-[(2-amino-4-{[(3S)-1-hydroxyhexan-3-yl]amino)-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
II) a malaria vaccine comprising an antigen comprising the sequence represented by SEQ ID NO: 1, SEQ ID NO: 2, SEQ ID NO: 3, or any of sequences substantially identical thereto.

3. A vaccine formulation for malaria comprising:
I) a pharmaceutical composition comprising:
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
II) a malaria vaccine comprising an antigen comprising the sequence represented by SEQ ID NO: 1, SEQ ID NO: 2, SEQ ID NO: 3, or any of sequences substantially identical thereto.

4. A kit comprising:
I) a pharmaceutical composition comprising:
i) (4E,8E,12E,16E,20E)-N-{2-[{4-[(2-amino-4-{[(3S)-1-hydroxyhexan-3-yl]amino}-6-methylpyrimidin-5-yl)methyl]benzyl}(methyl)amino]ethyl}-4,8,12,17,21,25-hexamethylhexacosa-4,8,12,16,20,24-hexaeneamide or a pharmaceutically acceptable salt thereof;
ii) squalane;
iii) an antioxidant agent A selected from the group consisting of ascorbate esters such as L-ascorbyl stearate and ascorbyl palmitate, mineral salts of ascorbic acid such as potassium ascorbate, sodium ascorbate, and calcium ascorbate, and ascorbic acid;
iv) an excipient selected from the group consisting of non-reducing sugars and sugar alcohols, except for mannitol;
v) a hydrophilic surfactant; and
vi) a lipophilic surfactant; and
II) a malaria vaccine comprising an antigen comprising the sequence represented by SEQ ID NO: 1, SEQ ID NO: 2, SEQ ID NO: 3, or any of sequences substantially identical thereto.

5. The method according to claim 1 or the combination drug according to claim 2, wherein the pharmaceutical composition is an oil-in-water type emulsion formulation or a lyophilized formulation thereof.

6. The method according to claim 1 or the combination drug according to claim 2, wherein the hydrophilic surfactant is polyoxyethylene sorbitan fatty acid esters (e.g., polysorbate 20, polysorbate 40, polysorbate 60, polysorbate 65, and polysorbate 80); polyoxyethylene hydrogenated castor oils (e.g., polyoxyethylene hydrogenated castor oil 10, polyoxyethylene hydrogenated castor oil 20, polyoxyethylene hydrogenated castor oil 40, polyoxyethylene hydrogenated castor oil 50, and polyoxyethylene hydrogenated castor oil 60); or polyoxyethylene polyoxypropylene glycols (e.g., polyoxyethylene (42) polyoxypropylene (67) glycol, polyoxyethylene (54) polyoxypropylene (39) glycol, polyoxyethylene (105) polyoxypropylene (5) glycol, polyoxyethylene (124) polyoxypropylene (39) glycol, polyoxyethylene (160) polyoxypropylene (30) glycol, polyoxyethylene (196) polyoxypropylene (67) glycol, and polyoxyethylene (200) polyoxypropylene (70) glycol).

7. The method according to claim 1 or the combination drug according to claim 2, wherein the hydrophilic surfactant is polysorbate 20, polysorbate 40, or polysorbate 80.

8. The method according to claim 1 or the combination drug according to claim 2, wherein the lipophilic surfactant is sorbitan fatty acid esters (e.g., sorbitan fatty acid ester, sorbitan monolaurate, sorbitan monopalmitate, sorbitan monostearate, sorbitan tristearate, sorbitan monooleate, sorbitan sesquioleate, sorbitan trioleate, and medium-chain triglyceride); glycerin fatty acid esters (e.g., glycerin fatty acid ester, glyceryl monostearate, glyceryl monomyristate, glyceryl monooleate, and glyceryl triisooctanoate); sucrose fatty acid esters (e.g., sucrose fatty acid ester, sucrose stearate, and sucrose palmitate); or propylene glycol fatty acid esters (e.g., propylene glycol fatty acid ester and propylene glycol monostearate).

9. The method according to claim 1 or the combination drug according to claim 2, wherein the lipophilic surfactant is sorbitan trioleate.

10. The method according to claim 1 or the combination drug according to claim 2, wherein the pharmaceutical composition further comprises an antioxidant agent B selected from the group consisting of tocopherols (e.g., α-tocopherol, β-tocopherol, γ-tocopherol, and δ-tocopherol); tocopherol acetate; and butylhydroxyanisole.

11. The method according to claim 1 or the combination drug according to claim 2, wherein the pharmaceutical composition further comprises α-tocopherol.

12. The method according to claim 1 or the combination drug according to claim 2, wherein the antioxidant agent A is ascorbyl palmitate, potassium ascorbate, sodium ascorbate, or ascorbic acid.

13. The method according to claim 1 or the combination drug according to claim 2, wherein the antioxidant agent A is sodium ascorbate or potassium ascorbate.

14. The method according to claim 1 or the combination drug according to claim 2, wherein the excipient is non-reducing sugars (e.g., sucrose and trehalose) or sugar alcohols (e.g., sorbitol, erythritol, xylitol, maltitol, and lactitol).

15. The method according to claim 1 or the combination drug according to claim 2, wherein the excipient is sucrose or trehalose.

16. The method according to claim 1 or the combination drug according to claim 2, wherein the increased amount in the area percentage value of an impurity UK-1.02 after a lyophilized formulation of the pharmaceutical composition is stored for 6 months at 5° C. is 5.0% or below.


SEQUENCE LISTING (3 sequences)

SEQ ID NO: 1 - protein, 371 aa, Artificial Sequence
  PfCSP4/38
AERSYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSGGSHHHHHH

SEQ ID NO: 2 - protein, 365 aa, Artificial Sequence
  PfCSP4/38
AERSYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSGGS

SEQ ID NO: 3 - protein, 358 aa, Artificial Sequence
  PfCSP4/38
YGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNS